(19) United States
(12) Patent Application Publication
(10) Pub. No.: US 2026/0229347 A1
(43) Pub. Date: Aug. 6, 2026

(54) IDENTITY-BASED SECURE MEDICAL DEVICE COMMUNICATIONS

(71) Applicant: ICU Medical, Inc., San Clemente, CA (US)

(72) Inventors: S. Sree Vivek, Chennai (IN); Hrishikesh Anil Dandekar, Pune (IN); Mark C. Rohlwing, Mesa, AZ (US); Chaitanya Mattur Srinivasamurthy, Lake Forest, IL (US)

(21) Appl. No.: 19/342,347

(22) Filed: Sep. 26, 2025

Related U.S. Application Data
(63) Continuation of application No. 17/648,652, filed on Jan. 21, 2022, now Pat. No. 12,431,238.
Application No. 17/648,652 is a continuation of application No. PCT/US2021/048262, filed on Aug. 30, 2021.
(60) Provisional application No. 63/108,141, filed on Oct. 30, 2020.

(30) Foreign Application Priority Data
Sep. 5, 2020 (IN) 202011038396

(51) Int. Cl. G16H 40/20; H04L 9/08; H04L 9/32

(52) U.S. Cl. CPC G16H 40/20; H04L 9/0869; H04L 9/0894; H04L 9/3263; H04L 2209/88

(57) ABSTRACT

The present disclosure is directed managing the operation of devices using identity-based cryptography. These techniques may include provisioning a master public key to each system that will communicate with a medical device using device-identifier specific cryptography. A master secret key is provisioned in a trusted processor of the medical device, and the medical device provisions its own device identifier-specific secret key using the master secret key. This setup facilitates several management features, including automatic initial configuration, signed logging, signed backup files, and secure binding of medication containers to the medical device.

### CROSS-REFERENCE TO RELATED APPLICATIONS

The present application is a continuation of U.S. patent application Ser. No. 17/648,652, filed on Jan. 21, 2022, which is a continuation of PCT Application No. PCT/US2021/048262, filed on Aug. 30, 2021 and titled “Identity-Based Secure Medical Device Communications,” which claims priority to Indian Provisional Patent Application No. 202011038396, filed on Sep. 5, 2020 and titled “Identity-Based Secure Medical Device Communications,” and U.S. Provisional Patent Application No. 63/108,141, filed on Oct. 30, 2020 and titled “Identity-Based Secure Medical Device Communications,” the contents of each of which are incorporated by reference herein and made part of this specification.

### TECHNICAL FIELD

This disclosure relates to the field of medical device management, and particularly to systems and methods for secure use of medical devices.

### BACKGROUND

Electronic medical devices often have processors and other computing components. Such medical devices may execute software and communicate with other computing systems via a network. Secure network communication may involve the encryption of communications transmitted from a medical device and/or decryption of communications received by the medical device. For example, a processor of the medical device may execute encryption/decryption operations on data to be transmitted via or received via the network.

### SUMMARY

Various techniques for managing the operation of devices using identity-based cryptography are described herein. These techniques may include provisioning a master public key to each system that will communicate with a medical device using device-identifier specific cryptography. A master secret key is provisioned in a trusted processor of the medical device, and the medical device provisions its own device identifier-specific secret key using the master secret key. This setup facilitates several management features, including automatic initial configuration, signed logging, signed backup files, and secure binding of medication containers to the medical device. These and other embodiments are described in greater detail below with reference to FIGS. 1-9. Although many of the examples are described in the context of medical devices, functions, and environments (including infusion pumps, medication dispensing functions, and hospital or clinical environments), the techniques described herein can be applied to other types of devices, functions, and environments.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an example cryptography setup system, medication preparation system, verification system, and secure medical device according to some embodiments.

FIG. 2 is a block diagram illustrating data flows and processing performed by components of a secure medical device during a secret key generation procedure according to some embodiments.

FIG. 3 is a block diagram illustrating data flows and interactions between a secure medical device and a verification system during an identity verification protocol according to some embodiments.

FIG. 4 is a block diagram illustrating data flows and interactions between a secure medical device and a verification system during a different identity verification protocol according to some embodiments.

FIG. 5 is a flow diagram of an illustrative routine for secure medication preparation according to some embodiments.

FIG. 6 is a flow diagram of an illustrative routine for secure medication administration according to some embodiments.

FIG. 7 is a block diagram illustrating data flows and processing performed during secure medication preparation and administration according to some embodiments.

FIG. 8 is a block diagram illustrating data flows and processing performed to securely manage logs and backup files according to some embodiments.

FIG. 9 is a block diagram illustrating data flows and processing performed to securely configure a secure medical device according to some embodiments.

### DETAILED DESCRIPTION OF ILLUSTRATIVE EMBODIMENTS

The present disclosure is directed to secure management of medical device communications using device identity-based encryption. A group of medical devices may each include a trusted processor in which the same master secret cryptographic key has been provisioned. The trusted processor is “trusted” in the sense that the master secret key is not accessible from outside of the trusted processor, and therefore the trusted processor may encrypt and decrypt data without exposing the master secret key. To generate a device identifier-specific secret cryptographic key for a medical device, the trusted processor executing within the medical device may use the master secret key and a device identifier uniquely associated with the medical device. Like the master secret key, each medical device stores their own device identifier-specific secret key such that it is not accessible from outside the trusted processor. Other devices and systems can communicate with a particular medical device using the device identifier for that medical device and a master public cryptographic key. The master public key is associated with the master secret key that has been provisioned in the trusted processors of each medical device. The master public key is also well-known or otherwise not kept secret among the devices and systems that communicate with the medical devices. Communications to a particular medical device can be encrypted using the device's identifier and the master public key such that only the device in possession of the device identifier-specific secret key generated using the same device identifier can decrypt the messages. Thus, other medical devices with different device identifiers cannot successfully decrypt the messages that have been encrypted using the device identifier, and devices that do not have access to the master secret key cannot derive the device identifier-specific secret key necessary to decrypt messages that are encrypted using the device identifier and master public key. In a similar manner, the medical device can sign, encrypt, and perform other cryptographic functions with the device identifier-specific secret key to ensure the authenticity of certain data, such as log files, backup files, and the like.

Some existing methods of secure communication require participating devices to exchange or agree on cryptographic keys. This pre-communication process may introduce a delay in the communications, and may expose the cryptographic keys to third parties thereby compromising the security of the communications. Identity-based encryption addresses some of these issues by deriving identifier-specific secret keys from a master secret key. In order to communicate securely with a particular device, a party need only know the identifier of the device and have access to the master public key that corresponds to the master secret key from which the identifier-specific secret key was derived. However, the device associated with the identifier must obtain the identifier-specific secret key for the device from a key generator. This may require network connection and communication with the key generator, and may expose the identifier-specific secret key during transmission. Moreover, even after the identifier-specific secret key is obtained by the device, it may be exposed to non-secure components of the device or to outside devices.

Some aspects of the present disclosure address the issues noted above, among others, by including a trusted processor in the medical devices for which device identifier-specific secret keys are generated. Within a particular medical device, the trusted processor may store the master secret key such that it is not accessible outside of the trusted processor. Moreover, the trusted processor may be configured to generate a device identifier-specific secret key for the medical device using the master secret key and a device identifier for the device. Thus, by including a trusted processor configured in this manner, the medical device effectively includes its own secret key generator and can self-provision a device identifier-specific secret key without requiring any communication with a separate secret key generator. Moreover, the device identifier may be any token that uniquely (or substantially uniquely) identifies the device, such as a serial number. Therefore, any number of medical devices may be manufactured with trusted processors and provisioned with the same master secret key. When deployed, each of the medical devices can self-provision their own unique device identifier-specific secret key that can be used to conduct secure communications and other secure data operations as described herein.

Additional aspects of the present disclosure relate to using device identifier-specific cryptography to verify the identity of a device. A verification system can initiate a procedure to verify the identity of a particular medical device by sending to the medical device verification data (e.g., a randomly-generated nonce) that has been encrypted using the medical device's identifier and the master public key. If the medical device successfully decrypts the verification data and sends it back to the verification system, then the medical device has proven that it has the device identifier-specific secret key that corresponds to the device identifier used to encrypt the data. In some embodiments, the verification system may send unencrypted verification data to the medical device, and the medical device may sign the verification data with its device identifier-specific secret key and return the signed verification data to the verification system. The verification system can then verify the signed verification data by decrypting it using the medical device's identifier and the master public key. In either implementation, the medical device may be considered to have proven its identity to the verification system assuming the following preconditions are satisfied: (1) the master secret key is not available outside of any of the medical devices' trusted processors, and (2) the trusted processors each self-provision their own device identifier-specific secret key based on the master secret key and their unique device identifier.

Further aspects of the present disclosure relate to using device identifier-specific cryptography to ensure medication is administered by only the specific medical device to which it is assigned. A medication preparation system may generate control data to assign a medication container to a specific medical device. The control data may be generated using the master public key and the device identifier for the medical device to which the medication container is to be assigned. The control data may be associated with the medication container such that it can be accessed by the medical device for authorization prior to administration. For example, the control data may be encoded into a visually reproducible and automatically readable form (e.g., a barcode on a label that is applied to the medication container) or encoded into electronic medium (e.g., a radio frequency identification or “RFID” tag of the medication container). Only the medical device to which the medication container has been assigned may decrypt the control data using the device identifier-specific secret key. Successful decryption and verification of the contents of the control data (e.g., medication identifier, patient identifier, device identifier, other data, or some combination thereof) may be required in order for the medical device to be operated to administer medication from the medication container.

Still further aspects of the present disclosure relate to using device identity-based cryptography to ensure the authenticity of medical device data, such as logs, backup files, and the like. A medical device may generate data regarding operation of the medical device, such as data regarding each medication administration procedure, each identification verification procedure, or the like. Such data may be logged for use in analyzing issues, such as those involving medication administered by the device (e.g., to detect overrides, administration irregularities, errors, and the like). In order to ensure the authenticity of such log data, the medical device may sign the log data using the device identifier-specific secret key. This signature may then be verified by any other system that has access to the master public key and the device identifier of the medical device. Successful verification of the signed log data ensures that the medical device associated with the device identifier is the source of the log data, and that the log data has not been altered after being signed by the medical device. A similar process may be used to encrypt or sign backup files, such as configuration backup files.

Additional aspects of the present disclosure relate to using identity-based cryptography to securely configure a medical device. An initial configuration system may provide a network connection—such as a Wi-Fi connection—to medical devices. In some embodiments, the medical devices may be configured to, upon initial startup after deployment, connect to the initial configuration system by default. When a medical device connects to the initial configuration system, the initial configuration system may begin an identity verification protocol. If the medical device proves its identity, then the initial configuration system may provide a configuration file to the medical device. The configuration file may include various configuration information to be used by the medical device, such as network connectivity information. In this way, medical devices can be automatically configured in a secure, verified manner.

Various aspects of the disclosure will now be described with regard to certain examples and embodiments, which are intended to illustrate but not limit the disclosure. Although aspects of some embodiments described in the disclosure will focus, for the purpose of illustration, on particular examples of medical devices, cryptographic key generation algorithms, and the like, the examples are illustrative only and are not intended to be limiting. In some embodiments, the systems and methods described herein may be applied to additional or alternative medical devices, cryptographic algorithms, etc.

### Overview of Example Setup Environment

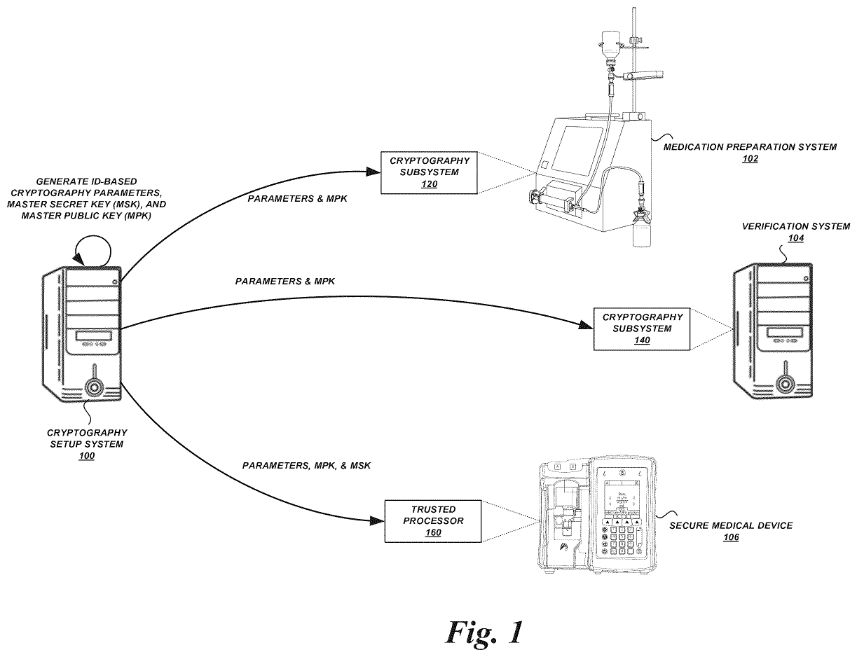

FIG. 1 illustrates an example environment in which a cryptography setup system 100 conducts a setup process to provision cryptographic keys, parameters, and the like to various devices that will be deployed in a network environment. As shown, the devices that are set up by the cryptography setup system 100 may include (but are not limited to) a medication preparation system 102, a verification system 104, and a secure medical device 106. Illustratively, the devices may be set up to be deployed in one or more healthcare facilities (e.g., hospitals) in which the secure medical device 106 is a medication administration device configured to administer medication prepared by the medication preparation system 102. In such an implementation, the verification system 104 may be (or be part of) an in-facility cloud-based system to manage use of the secure medical device 106, medication preparation system 102, etc. Although only one instance of a medication preparation system 102, verification system 104, and secure medical device 106 are shown in FIG. 1, in practice any number or combination of devices and systems may be set up by the cryptography setup system 100 and deployed to a network environment. For example, a single health care facility may have dozens, hundreds, or more individual secure medical devices 106 and/or medication preparation systems 102. The secure medical devices 106 may be the same as or different than each other, and the medication preparation systems 102 may be the same as or different than each other.

In some embodiments, the cryptography setup system 100 (also referred to simply as the “setup system”) may communicate with the medication preparation system 102, verification system 104, and secure medical device 106 via a communication network (also referred to simply as a “network”). The network may be a publicly-accessible network of linked networks, possibly operated by various distinct parties, such as the Internet. In some cases, the network may be or include a private network, personal area network, local area network, wide area network, global area network, cable network, satellite network, cellular data network, etc., or a combination thereof, some or all of which may or may not have access to and/or from the Internet. A medical device manufacturer, distributor, administrator, or other entity may operate the setup system 100 as described in greater detail below to provision cryptographic keys, cryptography algorithm parameters, and the like to the various systems and devices that are to be deployed. The cryptography setup system 100 may be a single physical computing device or logical association of multiple computing devices.

To perform the setup operation for devices to be deployed to a particular network environment, the cryptography setup system 100 may generate a set of data elements, including identity-based cryptography parameters, a master secret key, and a master public key. These data elements, once deployed, can be used to encrypt and decrypt data communicated between the devices that have been set up. More specifically, the encryption and decryption operations may be identity-based cryptographic operations that are based on an identifier of a particular device that is performing encryption or is to perform decryption. This feature ties the encryption/decryption operations to specific device identifiers, and therefore ties the operations to specific devices. For example, the cryptography parameters and master public key may be used with a device identifier to encrypt data that is sent to the device associated with the device identifier. The data encrypted in this manner may only be decrypted using a device identifier-specific secret key that has been generated using the same device identifier and parameters, and also the master secret key. A device that does not have access to the device identifier-specific secret key will therefore be unable (for practical purposes) to successfully decrypt the data. This aspect of device identifier-specific cryptography allows implementation of various identity verification and function authorization features as described in greater detail herein.

Table 1 below provides abbreviations that will be used herein for ease of description:

TABLE 1
Abbreviations

DID: device identifier
DISK: device identifier-specific secret key
MSK: master secret key
MPK: master public key
Params: identity-based cryptography parameters other than keys

In one specific, non-limiting embodiment, the cryptography setup system 100 may generate the params, MSK, and MPK using an algorithm such as that described in ISO/IEC 18033-5:2015, which is incorporated by reference herein. An example implementation is set forth in Table 2 below:

TABLE 2
Generation of Parameters and Master Keys

Establish base groups G₁, G₂, G₃ and a pairing e: G₁ × G₂ → G₃, where
G₁ and G₂ are in the form E(GF(q))[p] (an Elliptic curve over a finite
Galois Field of order p), where G₃ is a multiplicative group of order p,
where e is a bilinear pairing that maps two elements from G₁ and G₂ to
G₃, and where p and q are primes
Select a random generator Q in G₂, where Q is a primitive element in G₂
Generate the params as {Q, G₁, G₂, G₃, e}
Generate a random MSK in Z*ₚ, where Z*ₚ is the set of integers [1 to
p-1])
Generate the corresponding MPK as Q (MSK)

The medication preparation system 102, verification system 104, and secure medical device 106 may include various computing components, such as processors, network interface cards, volatile memory, long term storage, input/output components, displays, and the like. These systems and devices may also include components for performing device identity-based cryptography. For example, the medication preparation system 102 may include an identity-based cryptography subsystem 120, the verification system 104 may include an identity-based cryptography subsystem 140, and the secure medical device 106 may include a trusted processor 160. The identity-based cryptography subsystems 120, 140 may also be referred to simply as cryptography subsystems 120, 140, respectively. The cryptography subsystems 120, 140 and trusted processor 160 may be configured to perform cryptography using parameters and keys provided by the setup system 100.

As shown in FIG. 1, the setup system 100 may provide the parameters and MPK to the cryptography subsystems 120, 140 of the medication preparation system(s) 102 and/or verification system(s) 104, respectively. Due to the public nature of the MPK and the inability to decrypt data without a DISK that corresponds to the DID used to encrypt the data, the params and MPK may be made well-known to all devices that participate in identity-based cryptography communications. As long as the MSK and DISKs are kept secure, there may be no need for special security measures to maintain the parameters and MPK within the various devices.

The setup system 100 can also provide the parameters and MPK to the trusted processor 160 of each medical device 106 that is to be deployed in the same environment as the medication preparation system(s) 102 and/or verification system(s) 104. In addition, the setup system 100 can provide the MSK to the trusted processor 160 of each medical device 106. The trusted processor 160 may be implemented as a secure processor or a secure component of another processor. The trusted processor 160 is considered “secure” in the sense that it guarantees, for practical purposes, that the data stored inside is protected and not accessible from outside of the trusted processor 160. Thus, the MSK—and the DISK, once it is provisioned—in the trusted processor 160 are not accessible from outside the trusted processor 160, providing security for identity-based cryptography operations. The trusted processor 160 uses the DISK to generate output (e.g., output of an encryption/decryption operation). The output is provided to other portions of the secure medical device 106 or to other devices without exposing the DISK used to generate the output.

In some embodiments, the trusted processor 160 is a trusted platform module (“TPM”) that is a discrete hardware processor specifically designed to perform cryptographic operations. The TPM is physically isolated from the rest of the processing system of the secure medical device 106. For example, the TPM may be implemented on a discrete integrated circuit separate from other processing components of the secure medical device 106 (e.g., separate from the central processing unit and memory). In some embodiments, the trusted processor is a trusted execution environment (“TEE”) that is a component of a processor chipset (e.g., the chipset of the central processing unit). The TEE is therefore not physically isolated from the rest of the chipset, but may nevertheless be logically isolated such that access to data within the TEE is restricted.

Because each secure medical device 106 receives the MSK, each secure medical device 106 can dynamically self-provision their own DISK at a later time. As a result, secure medical devices 106 may be deployed without DISKs, and can self-provision their DISKs upon initial startup, as needed, or at some other time without requiring any additional data. Advantageously, this allows multiple medical devices to be deployed into an environment, potentially at different times, and without DISKs. These medical devices are nevertheless able to participate in identity-based cryptographic operations once they self-provision their own DISKs, and other systems and devices need only have access to the MPK and the publically-available DIDs of the medical devices.

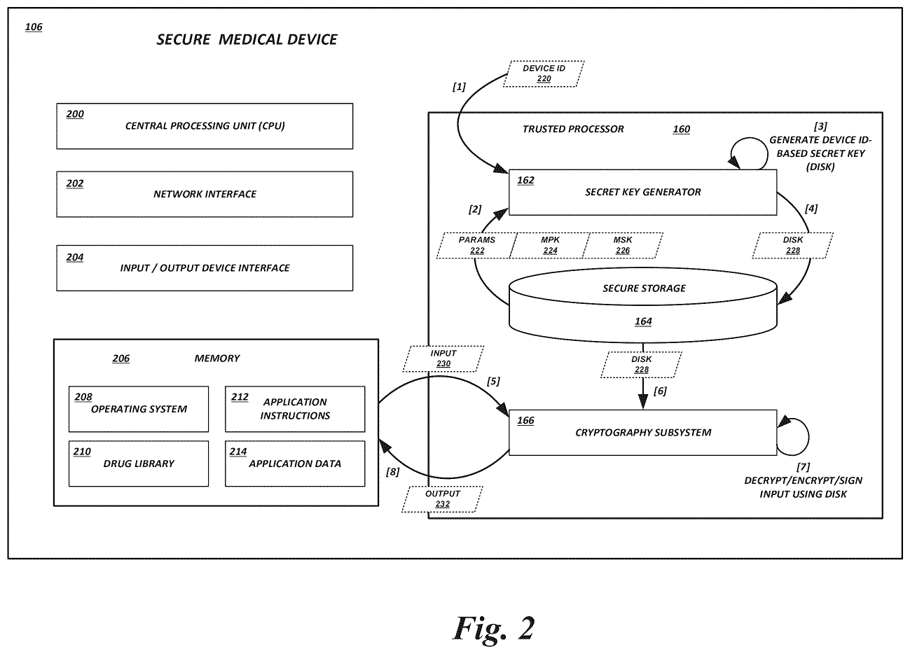

FIG. 2 illustrates an example of a secure medical device 106 showing interactions between various internal components of the secure medical device 106 to self-provision a DISK, and subsequent use of the DISK in identity-based cryptography operations. In some embodiments, as shown, the secure medical device 106 may include: one or more computer processors 200, such as physical central processing units (“CPUs”); one or more network interfaces 202, such as a network interface cards (“NICs”); one or more input/output device interfaces 204, and one or more computer readable memories 206, such as random access memory (“RAM”) and/or other non-transitory computer-readable media. The computer readable memory 206 may include computer program instructions that the computer processor 200 executes in order to implement one or more embodiments. For example, the computer readable memory 206 can store an operating system 208 that provides computer program instructions for use by the computer processor 200 in the general administration and operation of the secure medical device 106. The computer readable memory 206 may also include a drug library 210 with data regarding medications that may be administered using the secure medical device 106. The computer readable memory 206 may also include application instructions 212 and application data 214 for implementing medication administration operations, logging operations, and the like.

In some embodiments, the secure medical device 106 may be or include an infusion pump with various components to perform infusion pump operations. For example, an infusion pump may include a motor controller unit (“MCU”) configured to control a motor (not shown) that dispenses medication.

The secure medical device 106 includes a trusted processor 160. Although the trusted processor 160 is shown in FIG. 2 as being physically separate from other components of the secure medical device 106, in some embodiments the trusted processor 160 may be implemented as a discrete component of another processor as described above. The trusted processor 160 may include various physical or logical components. In some embodiments, as shown, the trusted processor 160 may include a secret key generator 162 to generate a DISK. The secret key generator 162 may be implemented in hardware, or as a combination of software and hardware to execute the software. The trusted processor 160 may include a secure storage 164 to store the parameters and keys used by the trusted processor 160. In some embodiments, the secure storage 164 may be a non-volatile secure element that stores the parameters and keys persistently, even after the medical device 106 is powered off. The trusted processor 160 may also include an identity-based cryptography subsystem 166 to perform identity-based cryptographic operations using the parameters and keys stored in the secure storage 164. The identity-based cryptography subsystem may be referred to simply as a cryptography subsystem 166.

The cryptography subsystem 166 may be implemented in hardware, or as a combination of software and hardware to execute the software.

### Device Identifier-Specific Secret Key Generation Process

The params 222, MPK 224, and MSK 226 that have been provisioned by the setup system 100 may be stored in the secure storage 164. To perform identity-based cryptography, the trusted processor may first self-provision the DISK 228 based on a unique or substantially-unique identifier of the secure medical device 106 or a component thereof. In some embodiments, some or all of the components of the secure medical device 106 may be associated with identifiers. For example, the processor 200 (or motherboard on which the processor 200 is located) may have a serial number assigned to it, and that unique serial number may serve as a DID 220 in the identity-based encryption and decryption processes described herein.

The trusted processor 160 may self-provision the DISK 228 in response to one or more events. For example, the first time the secure medical device 106 is powered on (or the first time the secure medical device 106 is powered on after being provisioned with the params 222, MPK 224, and MSK 226), the secret key generator 162 may generate the DISK. As another example, the secret key generator 162 may generate the DISK each time the secure medical device 106 is powered on, or each time a cryptographic operation is to be performed by the cryptography subsystem 166. As a further example, the DISK may be maintained in the secure storage 164 for a predetermined or dynamically determined period of time, after which it is removed. The secret key generator 162 may re-generate the DISK the next time it is to be used by the cryptography subsystem 166 or in response to some other event.

As shown in FIG. 2, at [1] the secret key generator 162 may obtain the DID 220. The DID 220 may be an identifier provided by a particular component (e.g., the processor 200), or retrieved from a particular storage location (e.g., storage in the basic input-output system or “BIOS” of the secure medical device 106). At [2] the secret key generator 162 may obtain from the secure storage 164 the params 222, MPK 224, and MSK 226 that have been provisioned by the setup system 100. At [3] the secret key generator 162 may generate the DISK using the DID 220, params 222, MPK 224, and MSK 226.

In one specific, non-limiting embodiment, the secret key generator 162 may generate the DISK using an algorithm such as that described in ISO/IEC 18033-5:2015, which is incorporated by reference herein. An example implementation is set forth in Table 3 below:

TABLE 3
Generation of Device Identifier-Specific Secret Key

Generate an identity element M = H₁(DID), where H₁ is a hash function
that maps identity strings to a group element, H₁: {0, 1}* → G₁
Generate DISK = MSK(M)

After the secret key generator 162 generates the DISK 228, the DISK 228 may be stored in the secure storage 164 at [4].

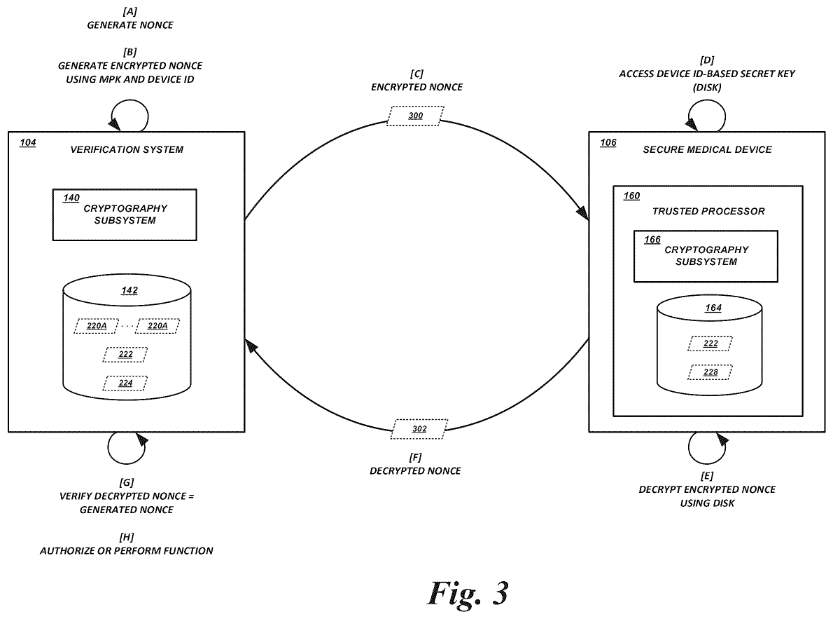

The cryptography subsystem 166 may use the DISK to perform encryption and/or decryption operations. At [5], the cryptography subsystem 166 may obtain input 230 to be encrypted or decrypted. For example, the verification system 104 may provide an encrypted nonce to the secure medical device 106 during an identification protocol, such as that shown in FIG. 3 and described in greater detail below. The nonce may have been encrypted using the MPK and the DID, and therefore may only be decrypted using the DISK that corresponds to the same DID.

At [6], the cryptography subsystem 166 may obtain the DISK 228 from the secure storage. At [7], the cryptography subsystem 166 may perform a cryptographic operation in the input 230 using the DISK 228. Returning to the example above, if the input 230 is an encrypted nonce, the cryptography subsystem 166 may decrypt the input 230 to generate decrypted output 232. At [8], the cryptography subsystem 166 may provide the output 232 outside of the trusted processor 160. For example, the output 232 may be provided to an application of the secure medical device 106 and stored in the application data 214.

### Identification Protocol

FIG. 3 is a block diagram of illustrative data flows and interactions during an identification protocol between a verification system 104 and a secure medical device 106. The identification protocol may be performed to verify the identity of the secure medical device 106. To do so, the verification system 104 may encrypt data using the DID of the secure medical device 106. Only if the secure medical device 106 has the DISK associated with the DID will the secure medical device 106 be able to decrypt the data and provide the unencrypted data back to the verification system 104.

At [A], the cryptography subsystem 140 or some other component of the verification system 104 can generate or otherwise obtain verification data to be used in the identification protocol. In some embodiments, the verification data may be a random data element, such as a randomly-generated nonce that is used only once. In some embodiments, the verification data may not be a nonce, but may instead be a meaningful value, such as a network address, another identifier, or the like.

At [B], the cryptography subsystem 140 can generate encrypted verification data using the verification data generated at [A], the params 222, MPK 224, and DID 220A associated with the secure medical device 106. In some embodiments, verification system 104 may have a data store 142 to store DIDs 220A-220N for each device with which the verification system 104 will perform the identification protocol. Params 222 and MPK 224 may also be stored in the data store 142. The data store 142 may be part of the cryptographic subsystem 140 or it may be a separate component or device of the verification system 104.

In one specific, non-limiting embodiment, a cryptography subsystem may encrypt data using an algorithm such as that described in ISO/IEC 18033-5:2015, which is incorporated by reference herein. An example implementation is set forth in Table 4 below, where Msg is the input to be encrypted (e.g., the verification data or some other plain text):

TABLE 4
Encryption Using Device Identifier and Master Public Key

```
Define H₁, H₂, H₃, H₄ as cryptographic hash functions:
  H₁: {0, 1}* → G₁, which maps an arbitrary string to an element in G₁
  H₂: G₃ → {0, 1}^{δ}, which maps an element in G₁ to a string of size δ
  H₃: {0, 1}^{δ} × {0, 1}^{δ} → Z*ₚ, which maps two strings of length δ to Z*ₚ
  H₃: {0, 1}^{δ} × {0, 1}^{δ}, which maps a string of length δ to another
Obtain an input Msg of length δ
Generate a δ-bit randomizer o using MPK, where “δ-bit randomizer o”
means o is a random string of length δ {0, 1}^{δ}
Generate an identity element M = H₁(DID)
Generate r = H₃(o, Msg)
Generate C₁ = rQ
Generate B = e(rM, MPK), where e is a bilinear pairing that maps two
elements from G₁ and G₂ to G₃
Generate C₂ = o ⊕ H₂ (B)
Generate C₃ = Msg ⊕ H₄ (o)
Generate output = (C₁, C₂, C₃)
```

At [C], the verification system 104 can send the encrypted verification data to the secure medical device 106. For example, if the verification data is a nonce and the encrypted verification data is an encrypted nonce 300, the verification system 104 can transmit the encrypted nonce 300 to the secure medical device 106.

At [D], the cryptography subsystem 166 of the secure medical device 106 can access the DISK 228 from the secure storage 164 to decrypt the encrypted verification data. At [E], the cryptography subsystem 166 can decrypt the encrypted verification data using the DISK 228.

In one specific, non-limiting embodiment, a cryptography subsystem may decrypt encrypted data using an algorithm such as that described in ISO/IEC 18033-5:2015, which is incorporated by reference herein. An example implementation is set forth in Table 5 below, where CT is the input to be decrypted (e.g., the encrypted verification data or some other cypher text):

TABLE 5
Decryption Using Device Identifier-Specific Secret Key

```
Define H₁, H₂, H₃, H₄ as cryptographic hash functions:
  H₁: {0, 1}* → G₁, which maps arbitrary string to an element in G₁
  H₂: G₃ → {0, 1}^{δ}, which maps an element in G₁ to a string of size δ
  H₃: {0, 1}^{δ} × {0, 1}^{δ} → Z*ₚ, which maps two strings of length δ to Z*ₚ
  H₃: {0, 1}^{δ} → {0, 1}^{δ}, which maps a string of length δ to another
Parse CT as a tuple {C₁, C₂, C₃}
Generate B = e(DISK, C₁), where e is a bilinear pairing that maps two
elements from G₁ and G₂ to G₃
Generate o = C₂ ⊕ H₂ (B)
Generate Msg = C₃ ⊕ H₄ (o)
Generate r = H₃(o, Msg)
Determine whether C₁ = rQ
  If yes, output Msg
  If no, output “error”
```

At [F], the secure medical device 106 can respond to the verification system 104 by sending the decrypted verification data to the verification system 104. For example, if the encrypted verification data was an encrypted nonce 300 and the secure medical device 106 successfully decrypted the encrypted nonce 300, the secure medical device 106 may transmit the decrypted nonce 302 to the verification system 104. In some embodiments, the secure medical device 106 may include additional information in the response. For example, the secure medical device 106 may include its DID 220A or other identifying information.

At [G], the verification system 104 can verify whether the response from the secure medical device 106 satisfies one or more identification verification criteria. An identification verification criterion may be a requirement that the response include a decrypted version of the encrypted verification data that was sent to the secure medical device 106 at [C]. For example, the verification system 104 may maintain a record of the unencrypted nonce generated at [A]. The record may associate the nonce with the specific secure medical device 106 (e.g., the record may reference the DID 220A of the secure medical device 106). Upon receipt of the decrypted nonce 302 from the secure medical device 106, the verification system 104 may access the record of the unencrypted nonce associated with the DID 220A of the secure medical device 106 from which the response was received, and compare the stored nonce to the decrypted nonce 302. If the decrypted nonce 302 matches the stored nonce, then the secure medical device 106 has proven that it has the DISK that corresponds the DID 220A. The medical device 106 may be considered to have proven its identity by virtue of proving that it has the DISK that corresponds to the DID 220A. In some embodiments, alternative or additional identification verification criteria may be required to be satisfied in order to complete the identification protocol successfully. For example, a timestamp may be stored in connection with the nonce 300, indicating when it was provided to the secure medical device 106. The medical device 106 may be required to respond within predetermined or dynamically determined threshold period of time after the time represented by the timestamp.

At [H], if the response from the secure medical device 106 has satisfied the one or more identification verification criteria, then the verification system 104 may authorize the secure medical device 106 to perform a function, or the verification system 104 may initiate a function. For example, the secure medical device 106 may be authorized to connect to a network, administer medication, receive a software update, receive a drug library update, initiate an override during a medication administration operation, or the like.

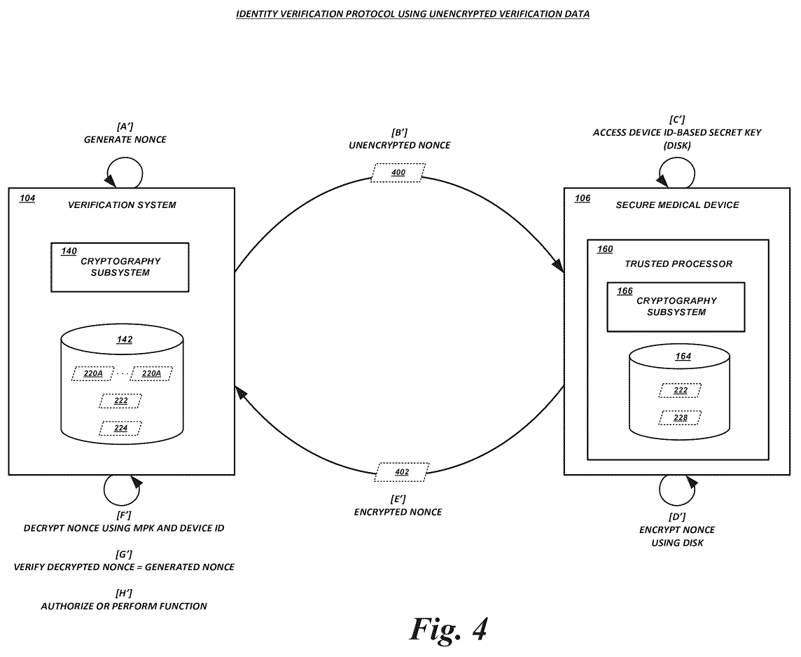

In some embodiments, the verification system 104 may not encrypt the verification data prior to transmitting it to the secure medical device 106. As shown in FIG. 4, the verification system 104 may generate verification data at [A′], and transmit the unencrypted verification data (e.g., the unencrypted nonce 400) to the secure medical device at [B′]. The verification system 104 does not necessarily get notified of—or otherwise determine in advance—the DID 220A of the secure medical device 106 to which the unencrypted verification data is sent. In this implementation, the secure medical device 106 may access the DISK at [C′], and encrypts the unencrypted verification data using the DISK at [D′] to generate encrypted verification data. The secure medical device 106 may transit a response to the verification system 104 at [E′]. The response includes the encrypted (signed) verification data, such as an encrypted nonce 402. In some embodiments, the response may also include the unencrypted verification data and/or the DID of the secure medical device 106. The verification system 104 decrypts the encrypted verification using the DID (either known in advance or provided by the secure medical device 106) and the MPK at [F′]. At [G′] the verification system 104 determines whether the response satisfies one or more identification verification criteria (e.g., the decrypted verification data matches the unencrypted verification data, either stored previously or included in the response from the secure medical device 106). At [H′], if the response has satisfied the one or more identification verification criteria, then the verification system 104 may authorize the secure medical device 106 to perform a function, or the verification system 104 may initiate a function.

### Identity-Based Medication Administration Authorization Process

Identity-based cryptography may be used to control which devices are able to perform operations using particular objects. In a health care setting, the devices may be secure medical devices 106, such as infusion pumps as described above. The objects may be medication containers, such as vials or intravenous (“IV”) fluid bags containing medical fluid that may be administered to patients using the secure medical devices. When a medication container is prepared for use, control information may be generated that assigns the medication container to a particular secure medical device 106 (or subset of secure medication devices 106). Advantageously, the control information may be encrypted using a DID of a medical device 106 to which the medication container is assigned. Only the medication device 106 with the DISK that corresponds to the DID will be able to decrypt the control information and administer the medication in the medication container. Thus, medication containers can be assigned to particular medication devices and only used by those secure medication devices 106; secure medication devices 106 may be prevented from using medication containers other than those to which they have been assigned.

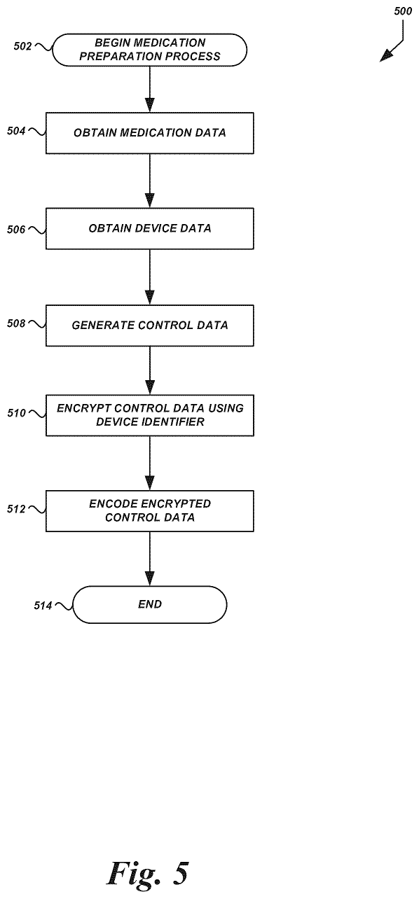

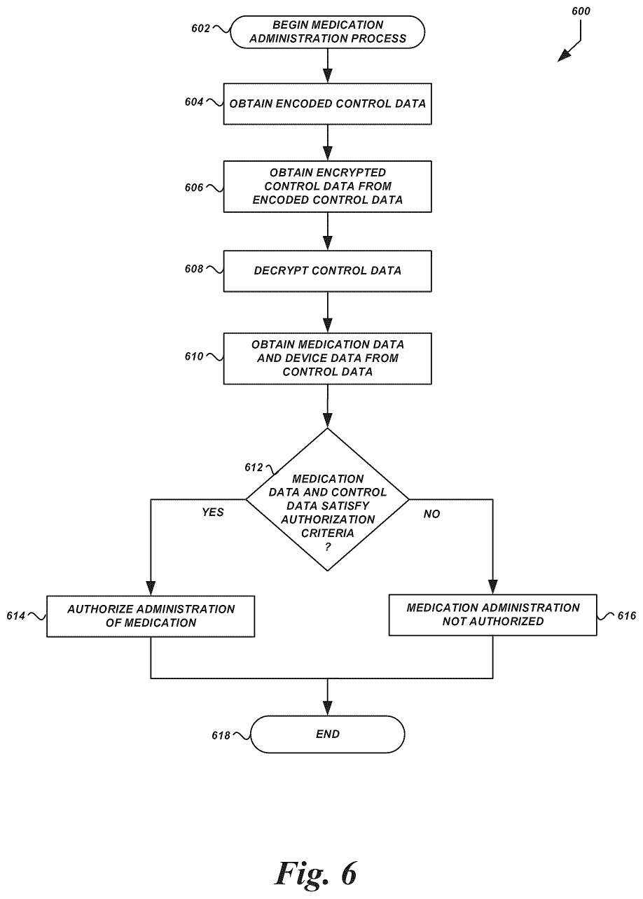

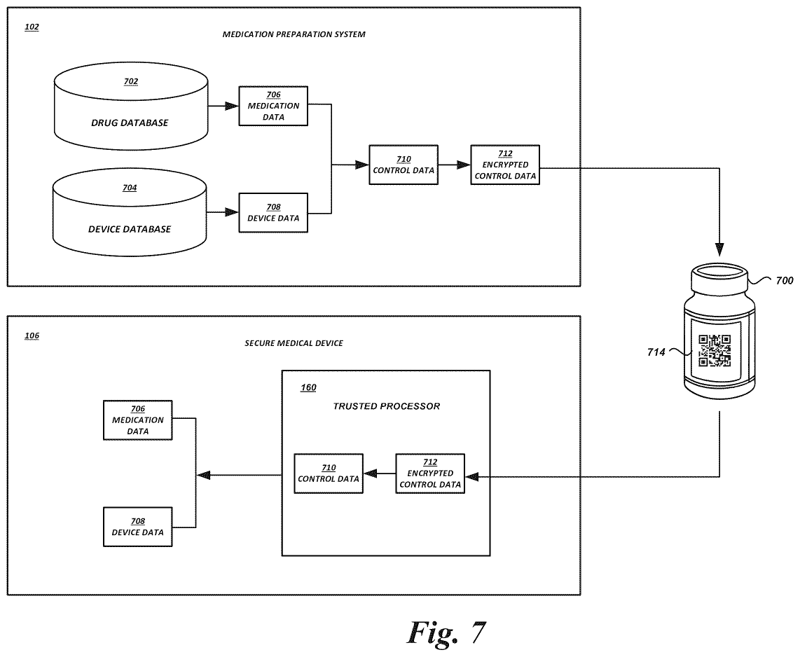

FIGS. 5 and 6 are flow diagrams of illustrative routines performed by the medication preparation system 102 and secure medical device 106, respectively, during an identity-based medication administration authorization process. The routines 500 and 600 of FIGS. 5 and 6 will be described with further reference to FIG. 7, which is a block diagram illustrating the generation of control information by a medication preparation system 102 to assign a medication container 700 to a particular secure medical device 106.

The routine 500 shown in FIG. 5 begins at block 502. The routine 500 may begin in response to an event, such as when a pharmacist or medication preparation technician accesses a software application of the medication preparation system 102 to initiate preparation of a medication container for administration by a particular secure medical device 106. When the routine 500 is initiated, a set of executable program instructions stored on one or more non-transitory computer-readable media (e.g., hard drive, flash memory, removable media, etc.) may be loaded into memory (e.g., random access memory or “RAM”) of the medication preparation system 102. The executable instructions may be executed by a hardware-based computer processor (e.g., a central processing unit or “CPU”) of the medication preparation system 102. In some embodiments, the routine 500 or portions thereof may be implemented on multiple processors, serially or in parallel.

At block 504, the medication preparation system 102 may obtain medication data 706 regarding the medication that is in (or is being compounded into) the medication container 700. The medication data 706 may include an identifier of the mediation, a name of the medication, dosing information, other data, or some combination thereof. In some embodiments, the medication data 706 may be obtained from a drug database 702 of the medication preparation system 102.

At block 506, the medication preparation system 102 may obtain device data 708 regarding the secure medical device 106 to which the medication container is being assigned. The device data 708 may include the DID and/or other data regarding the secure medical device 106. In some embodiments, the device data 708 may be obtained from a device database 704 of the medication preparation system 102.

At block 508, the medication preparation system 102 generate control data 710 using the medication data 706 and device data 708. The control data 710 may be a data structure comprising a data element for the medication data 706 and a data element for the device data 708. In some embodiments, the device data 708 may be concatenated to the medication data 706 to generate the control data 710. For example, the first m bytes of control data 710 may be reserved for the medication data 706, and the next n bytes may be reserved for device data 708 (or vice versa). As another example, the portion of control data 710 preceding a particular character or value may be medication data 706, and next portion of control data 710 may be device data 708 (or vice versa). In some embodiments, the control data 710 may also include other data, such as patient data (e.g., an identifier of the patient to whom the medication is prescribed), unit data (e.g., an identifier of the unit of the health care facility in which the medication may ne administrated), etc. The additional data may be included in the control data 710 at other locations in the same manner as the medication data 706 and device data 708. The example structures of control data 710 described herein are illustrative only, and are not meant to be limiting. In some embodiments, other configurations of control data 710 may be used. In some embodiments, the control data 710 may not include both medication data 706 and device data 708, but may instead include medication data 706 or device data 708 (and, optionally, other data such as patient data).

At block 510, the cryptography subsystem 120 of the medication preparation system 102 may generate encrypted control data 712 by encrypting the control data 710 using the DID of the secure medical device 106 to which the medication container 700 is assigned. In some embodiments, the encryption operation may be performed using the encryption algorithm set forth above.

At block 512, the cryptography subsystem 120 or some other component of the medication preparation system 102 may encode the encrypted control data 712 into a form that may be represented by a control item 714. The control item 714 may be coupled to or integrated with the medication container 700, and may include the encoded control data. For example, the control item 714 may be a label and the encoded control data may be a barcode or quick response (“QR”) code. In this case, the medication preparation system 102 may encode the encrypted control data 712 into a barcode or QR code, and the encoded control data may be printed onto the label such that it may be scanned by the secure medical device 106 to recover the encrypted control data 712. in some embodiments, the control item may be an electronic data store accessible via wired or wireless communication. For example, the control item may be a radio frequency identification (“RFID”) tag or a microchip. The encrypted control data may be encoded into a form that is stored by control item and accessible from the control item by the secure medical device 106. The process 500 may terminate at block 514.

Once the control item 712 has been provisioned with encoded control data and affixed to the medication container 700 (if necessary), the medication container 700 may be delivered for use by the secure medical device 106. The secure medical device 106 may then perform a routine, such as routine 600 sown in FIG. 6, to read and process the control data and determine whether to proceed with administration of the medication within the medication container 700.

The routine 600 begins at block 602. The routine 600 may begin in response to an event, such as when a health care provider accesses a software application of the secure medical device 106 to initiate administration of medication from the medication container 700. When the routine 600 is initiated, a set of executable program instructions stored on one or more non-transitory computer-readable media (e.g., hard drive, flash memory, removable media, etc.) may be loaded into memory (e.g., random access memory or “RAM”) of the secure medical device 106. The executable instructions may be executed by one or more hardware-based computer processors of the secure medical device 106, such as the processor 200 and trusted processor 160.

At block 604, the secure medical device 106 may obtain the encoded control data from the control item 714 of the medication container 700. If the control item 714 is a label with a barcode or QR code, a scanning device may be used to read the encoded control data and provide it—in encoded or decoded form—to the secure medical device 106. Other types of control items may be accessed depending upon the implementation (e.g., an RFID tag may be read by an RFID reader, a microchip may be read by a chip reader, etc.).

At block 606, the secure medical device 106 may obtain the encrypted control data 712 from the encoded control data, if the encoded control data is read from the control item 714 without being automatically decoded as part of the reading process. For example, a barcode may be automatically decoded when read by a barcode reader such that decoded data represented by the barcode (encrypted control data 712 in this example) is output by the barcode reader to other components of the secure medical device 106. If such decoding is not performed automatically, the secure medical device 106 may be configured (e.g., via application instructions 212) to perform the necessary decoding to recover the encrypted control data 712 represented by the encoded control data read from the control item 714.

At block 608, the trusted processor 160 of the secure medical device 106 may generate decrypted control data 710 from the encrypted control data 712. The cryptography subsystem 166 may obtain the DISK 228 from the secure storage 164, and use the DISK 228 to decrypt the encrypted control data 712. In some embodiments, the decryption operation may be performed using the algorithm set forth above.

At block 610, the secure medical device 106 may obtain medication data 706 and device data 708 from the decrypted control data 710. The secure medical device 106 may be configured (e.g., via application instructions 212) to parse the medication data 706 and device data 708 from the control data 710. For example, the first m bytes of decrypted control data 710 may be reserved for the medication data 706, and the next n bytes may be reserved for device data 708 (or vice versa). As another example, the portion of control data 710 preceding a particular character or value may be medication data 706, and next portion of control data may be device data 708 (or vice versa). In some embodiments, as described above, the control data 710 may also include other data, such as patient data (e.g., an identifier of the patient to whom the medication is prescribed), unit data (e.g., an identifier of the unit of the health care facility in which the medication may ne administrated), etc. The additional data may be included in the control data 710 at other locations in the same manner as the medication data 706 and device data 708. The example structures of control data 710 described herein are illustrative only, and are not meant to be limiting. In some embodiments, other configurations of control data 710 may be used. In some embodiments, the control data 710 may not include both medication data 706 and device data 708, but may instead include medication data 706 or device data 708 (and, optionally, other data such as patient data).

At decision block 612, the secure medical device 106 may determine whether the medication data 706 and device data 708 satisfy one or more authorization criteria to authorize administration of the medication. The one or more authorization criterion may include: successfully decrypting the encrypted control data and obtaining the control data therefrom; determining that a DID from the device data 708 matches the DID 220 of the current secure medical device 106; determining that a medication ID from the medication data 706 matches a medication in a drug library 210 of the secure medical device 106; determining that the medication represented by the medication data 706 is permitted to be administered by the secure medical device 106; determining that a patient ID from patient data matches a particular patient ID (e.g., a patent ID of a patient whose admittance bracelet or other medical identification has been scanned or entered); other authorization criteria; or any combination thereof. If the one or more authorization criteria have been satisfied, the routine 600 may proceed to block 614, wherein the medication administration operation is authorized and proceeds. Otherwise, if an authorization criterion has not been satisfied, the routine 600 may proceed to block 616 where an unauthorized state is entered. The process 600 may terminate at block 618.

### Logging and Backup Verification

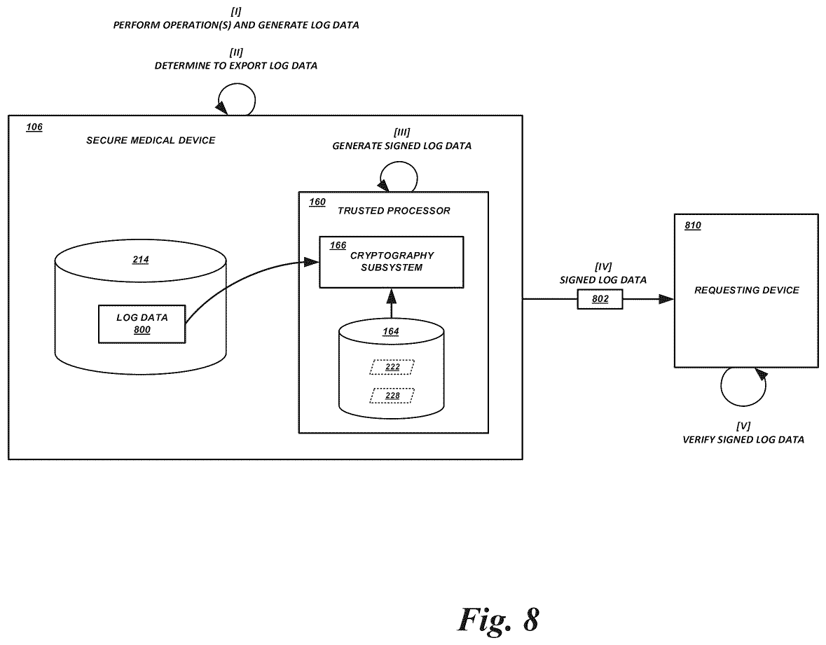

FIG. 8 is a block diagram of illustrative data flows and operations during log file generation, export, and consumption. During operation, a secure medical device 106 may generate log data regarding the functions that the secure medical device 106 performs. For example, the secure medical device 106 may generate one or more records regarding a medication administration process, such as the medication administration process described above. Such records may be stored in a log file. Under certain circumstances, a forensic analysis of the log file may be performed, such as when an issue with a particular medication administration process occurs. In order to ensure that the logged record of the process is an authentic, accurate record of the operations performed by the secure medication device 106, the log file data may be signed using the DISK before being exported by—or otherwise provided outside of—the secure medical device 106. Advantageously, the signing of log data using the DISK facilitates verification, by a third party, of the authenticity of the source of the log data and the integrity of the data within the log.

At [I], the secure medical device 106 performs various operations and generates log data 800 regarding the operations. The log data 800 may be stored within the secure medical device 106, such as the application data 214 area of memory, in a persistent data store, or the like.

At [II], the secure medical device 106 may determine that log data 800 is to be exported. In some embodiments, the secure medical device 106 may receive a request or a command to export the log data. In some embodiments, the secure medical device 106 may export the log data 800 on a predetermined or dynamically determined schedule, or in response to some other event. The log data 800 to be exported may be all log data currently stored in the secure medical device 106, the log data that has been added since the last export, or a subset of log data associated with the current export process (e.g., log data satisfying a filter or other criteria specified in the request or export configuration).

At [III], the cryptography subsystem 166 may obtain the DISK 228 from the secure data store 164 and sign the log data 800 to be exported. To sign the log data 800, the cryptography subsystem 166 may encrypt the log data 800 using the DISK 228. The signed data 802 may also include an unencrypted version of the log data 800. Thus, a recipient of the signed log data 802 can decrypt the encrypted version using the MPK and the DID associated with the secure medical device 106, and determine whether the decrypted log data matches the unencrypted log data. If so, the unencrypted log data is considered to be authentic.

At [IV], the secure medical device 106 can export the signed log data 802 to a third-party device or system. For example, as shown, the signed log data 802 may be exported to a requesting device 810. In some embodiments, the signed log data 802 may be exported to an intermediary data store, where it is stored and accessible to third party devices or systems.

At [V], the requesting device 810 (or some other third-party deice or system) can decrypt the signed data 802 using the MPK and the DID associated with the secure medical device 106. The requesting device 810 may then compare the decrypted singed data to the unencrypted log data. If the two data sets match, then the requesting device 810 can be assured that the log data did originate from the secure medical device 106 and has not be tampered with.

In some embodiments, data other than log data may be signed by the secure medical device 106 before being exported. For example, the secure medical device 106 may generate a backup of its configuration settings, such as data regarding networks and devices with which the secure medical device 106 is to interact. In order to ensure the authenticity of the configuration data and that it has not been tampered with, the secure medical device 106 may sign the backup file before exporting the backup file for storage. Subsequently, when the secure medical device 106 is to restore a backup file, the secure medical device 106 can decrypt the signed data using its own DID and the MPK. If the decrypted data 106 matches the unencrypted data, then the backup file is considered to be authentic and not tampered with.

### Initial Communication and Configuration

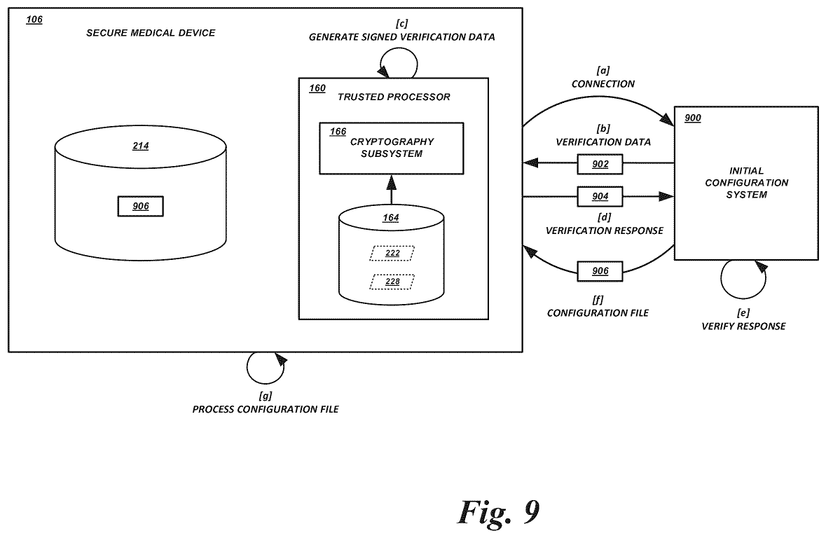

FIG. 9 is a block diagram of illustrative data flows and operations during an initial communication and configuration of the secure medical device 106. The secure medical device 106 may be configured to, upon initial startup after deployment, connect by default to an initial configuration system an obtain a configuration file. The configuration file may include various configuration information to be used by the medical device 106, such as network connectivity information. In this way, the medical device can be automatically configured in a secure, verified manner.

At [a], the secure medical device 106 connects to the initial configuration system 900. The initial configuration system 900 may include a verification system 104 or vice versa. In some embodiments, the initial configuration system 900 may be a separate device or system from the verification system 104. The initial configuration system may provide a network connection—such as a Wi-Fi connection—for secure medical devices to connect to for configuration data.

At [b], the initial configuration system 900 responds by initiating an identity verification protocol, such as a one of the protocols described above. For example, the initial configuration system 900 may generate verification data such as a random nonce 902, and send the nonce to the secure medical device 106. The secure medical device 106 may sign the nonce 902 using the DISK 228 at [c], and respond to the initial configuration system 900 by sending back the signed nonce 904 at [d]. In some embodiments, the response may include additional data, such as the DID of the secure medical device 106.

The initial configuration system 106 may decrypt the signed response using the DID and the MPK. If the signed response satisfies one or more identification criteria at [e], the initial configuration system 900 may determine that secure medical device 106 has successfully proven its identity. In response, the initial configuration system 106 may provide a configuration file 904 to the secure medical device 106 at [f]. The secure medical device 106 may process the configuration file 904 at [g]. For example, the secure medical device 106 may read configuration settings (e.g., a network address of a system to which the medical device 106 is to connect with) from the configuration file 904 and the settings during operation.

### OTHER CONSIDERATIONS

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a computer processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A computer processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A system comprising:
a plurality of medical devices, wherein a medical device of the plurality of medical devices comprises:
a trusted processor comprising:
a secure data store storing a master secret key and a master public key;
a secret key generator configured to:
generate a device identifier-specific secret key using the master secret key and an identifier uniquely associated with the medical device; and
store the device identifier-specific secret key in the secure data store; and
a cryptography subsystem configured to:
receive encrypted data; and
generate decrypted data using the encrypted data and the device identifier-specific secret key;
wherein the trusted processor is configured to prohibit output, from the trusted processor, of the device identifier-specific secret key and the master secret key; and
a setup system comprising computer-readable memory and one or more computer processors, wherein the setup system is configured to:
generate the master secret key and the master public key; and
provision the master secret key and the master public key to the trusted processor of each of the plurality of medical devices.

2. The system of claim 1, further comprising a verification system comprising computer-readable memory and one or more computer processors, wherein the verification system is configured to:
generate a random nonce;
encrypt the random nonce to generate an encrypted nonce based at least partly on the master public key and an identifier of the medical device
send the encrypted nonce to the medical device;
receive a response from the medical device; and
determine whether the response satisfies one or more identification verification criteria.

3. The system of claim 1, further comprising a verification system comprising computer-readable memory and one or more computer processors, wherein the verification system is configured to:
generate a random nonce;
send the random nonce to the medical device;
receive a response from the medical device, wherein the response comprises encrypted data;
decrypt the encrypted data using the master public key and an identifier of the medical device; and
determine whether the response satisfies one or more identification verification criteria.

4. The system of claim 1, further comprising an initial configuration system comprising computer-readable memory and one or more computer processors, wherein the initial configuration system is configured to:
execute an identification verification protocol with the medical device based at least partly on the master public key and an identifier of the medical device; and
in response to successful completion of the identification verification protocol, send a configuration file to the medical device.

5. The system of claim 1, further comprising a medication preparation system comprising computer-readable memory and one or more computer processors, wherein the medication preparation system is configured to:
generate control data comprising:
a device identifier associated with the medical; and
a medication identifier associated with a medication container;
generate encrypted control data using the control data, the master public key, and the device identifier; and
generate an encoded label for the medication container using the encrypted control data.

6. The system of claim 1, wherein the medical device is further configured to:
generate log data;
sign the log data using the cryptography subsystem to generate signed log data; and
export the signed log data to a third-party device.

7. The system of claim 1, wherein the medical device is further configured to:
generate a configuration backup file;
sign the configuration backup file using the cryptography subsystem to generate a signed configuration backup file; and
export the signed configuration backup file.

8. An infusion pump comprising:
a trusted processor comprising:
a secure data store storing a master secret key;
a secret key generator configured to:
generate an identifier-specific secret key using the master secret key and an identifier associated with the infusion pump; and
store the identifier-specific secret key in the secure data store; and
a cryptography subsystem configured to:
receive encrypted data; and
decrypt the encrypted data using the identifier-specific secret key; and
a motor configured to cause fluid to be administered from a medication container.

9. The infusion pump of claim 8, further comprising an input device,
wherein the input device is configured to:
read encoded control data from the medication container; and
decode the encoded control data to obtain encrypted control data; and
wherein the cryptography subsystem is configured to:
receive the encrypted control data; and
decrypt the encrypted control data to generate decrypted control data based at least partly on the identifier-specific secret key, wherein the decrypted control data comprises a control device identifier.

10. The infusion pump of claim 9, wherein the motor is permitted to be activated based at least partly on the control device identifier matching the identifier associated with the infusion pump.

11. The infusion pump of claim 8, further comprising a network interface configured to receive a nonce from a verification system,
wherein the cryptography subsystem is further configured to encrypt the nonce using the identifier-specific secret key to generate an encrypted nonce, and
wherein the network interface is further configured to send the encrypted nonce to the verification system.

12. The infusion pump of claim 8, further comprising a network interface configured to receive an encrypted nonce from a verification system,
wherein the cryptography subsystem is further configured to decrypt the nonce using the identifier-specific secret key to generate a decrypted nonce, and
wherein the network interface is further configured to send the decrypted nonce to the verification system.

13. The infusion pump of claim 8, further comprising a network interface configured to:
execute an identification verification protocol with an initial configuration system based at least partly on the identifier-specific secret key; and
in response to successful completion of the identification verification protocol, receive a configuration file from the verification system.

14. The infusion pump of claim 8, further configured to:
generate log data;
sign the log data using the cryptography subsystem to generate signed log data; and
export the signed log data to a third-party device.

15. The infusion pump of claim 8, further configured to:
generate a configuration backup file;
sign the configuration backup file using the cryptography subsystem to generate a signed configuration backup file; and
export the signed configuration backup file.
